(19) United States
(12) Patent Application Publication
(10) Pub. No.: US 2023/0203560 A1
(43) Pub. Date: Jun. 29, 2023

(54) GLUCOSYL ESTERS FOR INFECTION SCREENING

(71) Applicant: Board of Regents, The University of Texas System, Austin, TX (US)

(72) Inventors: HUA-YU WANG, San Antonio, TX (US); STANTON F. MC HARDY, San Antonio, TX (US); WALDEMAR GORSKI, San Antonio, TX (US); MICHAEL BEKHIT, San Antonio, TX (US)

(21) Appl. No.: 17/927,884

(22) PCT Filed: May 27, 2021
(86) PCT No.: PCT/US2021/034392

§ 371 Date: Nov. 27, 2022

Related U.S. Application Data
(60) Provisional application No. 63/030,458, filed on May 27, 2020.

(51) Int. Cl. C12Q 1/44; C07H 13/04

(52) U.S. Cl. CPC C12Q 1/44; C07H 13/04

(57) ABSTRACT

Compositions and methods of use for a glucosyl ester or a salt or solvate thereof, to detect and/or measure LE activity in a sample are disclosed.

### PRIORITY

This Application claims priority to U.S. Provisional Application Ser. No. 63/030,458 filed May 27, 2020, which is incorporated herein by reference in its entirety.

### STATEMENT REGARDING FEDERALLY FUNDED RESEARCH

None

### BACKGROUND OF THE INVENTION

### A. Field of the Invention

The invention generally concerns compositions, devices, kits, and methods for detecting the presence of leukocyte esterase. In particular aspects, the invention concerns glucosyl esters substrates of leukocyte esterase (LE) and compositions, devices and kits including or using the same.

### B. Description of Related Art

Every day, thousands of patient samples are tested in clinical laboratories for infection. However, over half of them are not infected meaning that they were unnecessarily sent for testing (Alexander B. Critical Values, 2012, 5, 6-8; Messacar et. al., J. Clin. Microbiol. 2017, 55, 715-723). More reliable screening is needed before sending samples to diagnostic labs for a more complete analysis. The benefits of such a screening will include saving resources, minimizing patient suffering, and reducing the excessive use of antibiotics and related growth of lethal antibiotic-resistant bacteria, which is one of the major challenges of modern medicine.

Historically, the diagnosis of infections in biological samples has relied on microbial cultures and non-culture methods including microscopic staining, counting leukocytes under a microscope, antibody and antigen immunoassays, and nucleic acid amplification testing (Washington et. al., Lab. Med. 1981, 12, 294-296; Carroll et. al., Am. J Clin. Pathol. 1994, 101, 100-103; McCabe et. al., Arthritis Rheumatol. 2017, 69, 103-107; Liao et al., J. Clin. Microbiol. 2006, 44, 561-570; Pan et al., Biosens. Bioelectron. 2010, 26, 649-654; Cartwright et al., Clin. Microbiol. Newsl. 1994, 16, 33-40). These are labor-intensive techniques, which require specialized instrumentation and trained personnel.

The enzyme leukocyte esterase (LE) is an important biomarker for diagnosing and monitoring infections, a common and often devastating clinical problem. However, the development of novel LE assays has been limited (Kotani et al., Clinica Chim. Acta, 2014, 433:145-49; Murthy and Karmen, Biochem. Med. Metabol. Bio., 1988, 40:260-68; Mastropaolo and Yourno, Anal. Biochem. 1981, 115:188-93; Johnson and Schaeper, Bioconjugate Chem. 1997, 8: 76-80) and the relevant literature is dominated by the studies of clinical utility of existing LE kits and strips (McNabb et al., J. Arthroplasty 2017, 32:220-22; Colving et al., Skeletal Radiol. 2015, 44:673-77; Yadav et al., Int. J. Pharm. Bio. Sci. 2015, 6B:370-75; Ducharme et al., Can. J. Emergen. Med. 2007, 9:87-92; Bimstein et al., Pediatr. Dentist. 2004, 26:310-15), which are all based on optical assays. While such assays have been fairly useful, they often provide only semi-quantitative readings and have a limited resolution (especially, in color or opaque media).

Examples of commercial leukocyte esterase reagent strips are CHEMSTRIP® 9 and CHEMSTRIP® LN (both sold by Bio-Dynamics, Indianapolis, Ind.); and MULTISTIX® 2 Reagent Strips and AMES LEUKOSTIX® (both available from Ames, Division of Miles Laboratory, Elkhart, Ind.). Techniques for using these commercial leukocyte esterase reagent strips are well known from their use for in vitro urine analysis (e.g., Scheer, Am. J. Clin. Pathol., 1987, 87:86-93).

All of these commercial leukocyte esterase reagent strips contain an indoxyl carbonic acid ester which is hydrolyzed to indoxyl by leukocyte esterase. The indoxyl thus formed reacts with a diazonium compound in the strip to produce a color which indicates the presence of the leukocyte esterase. The degree of darkening of the strips is a semi-quantitative indication of the amount of leukocyte esterase present in a sample. Such colorimetric determination cannot be effectively used in turbid or color samples, e.g., bloody biological fluids, and it often requires toxic chromogenic agents, multiple liquid-handling steps, and time-consuming incubation.

### SUMMARY OF THE INVENTION

Compounds, compositions, kits, and devices of the current invention provide a solution to the problems associated with current colorimetric LE assays. In particular, the glucosyl esters described herein provide for detection of LE activity in a relatively quick and more sensitive and quantitative manner. The glucosyl esters described herein can be cleaved by LE and release glucose in an amount proportional to the enzymatic activity of LE. The glucose released can be detected (e.g., electrochemically) with a glucose detection component. Selectivity, sensitivity, and short analysis time are some of the benefits gained using assays described herein.

A class of glucosyl esters are described herein that enable detection of LE by detecting glucose in a sample. Detection methods described herein overcome the limits of optical assays and open the prospect of point-of-care devices for the rapid quantification of infection. In particular, the inventors describe the synthesis of the glucosyl esters substrates for LE, their enzymatic kinetics, and their performance in the development of LE assays.

Certain embodiments are directed to glucosyl ester substrates for LE. In certain aspects, the glucosyl ester can have a chemical formula of Formula I or a salt (e.g., pharmaceutically acceptable salt) or solvate thereof:

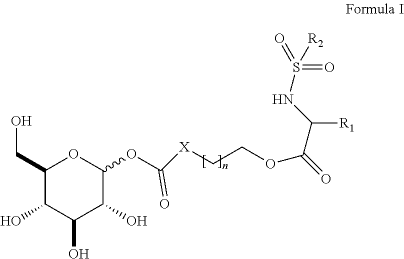

wherein, X can be CR₃R₄ or NR₅, n can be 1, 2, 3, 4, 5 or 6 and R₁, R₂, R₃, R₄ and R₅ can independently be hydrogen, a C₁ to C₈ alkyl, a substituted C₁ to C₈ alkyl, a C₃ to C₇ cycloalkyl, a substituted C₃ to C₇ cycloalkyl, a heteroalkyl, a substituted heteroalkyl, a heterocycle, a substituted heterocycle, an aryl, a substituted aryl, a fused aryl, a substituted fused aryl, a heteroaryl, or a substituted heteroaryl. The substituted C₁ to C₈ alkyl can be substituted with 1 to 3 halogens, a C₁ to C₃ alkyl, OH, NH₂, C(O)NH₂, CO₂H, CH₂OH and —C(O)NHR′, C(O)NR′₂, OR′, CO₂R′, CH₂OR′, NHR′, N(R′)₂, or combinations thereof, wherein R′ is a C₁ to C₃ alkyl or a halogenated alkyl. The substituted C₃ to C₇ cycloalkyl can be substituted with 1 to 3 halogens, a C₁ to C₃ alkyl, OH, NH₂, C(O)NH₂, CO₂H, CH₂OH and —C(O)NHR′, C(O)NR′₂, OR′, CO₂R′, CH₂OR′, NHR′, N(R′)₂, or combinations thereof, wherein R′ is a C₁ to C₃ alkyl or a halogenated alkyl. The substituted aryl can be substituted with 1 to 3 halogens, a C₁ to C₃ alkyl, OH, NH₂, C(O)NH₂, CO₂H, CH₂OH and —C(O)NHR′, C(O)NR′₂, OR′, CO₂R′, CH₂OR′, NHR′, N(R′)₂, or combinations thereof, wherein R′ is a C₁ to C₃ alkyl or a halogenated alkyl. The substituted fused aryl can be substituted with 1 to 3 halogens, a C₁ to C₃ alkyl, OH, NH₂, C(O)NH₂, CO₂H, CH₂OH and —C(O)NHR′, C(O)NR′₂, OR′, CO₂R′, CH₂OR′, NHR′, N(R′)₂, or combinations thereof, wherein R′ is a C₁ to C₃ alkyl or a halogenated alkyl. The substituted heteroaryl can be substituted with 1 to 3 halogens, a C₁ to C₃ alkyl, OH, NH₂, C(O)NH₂, CO₂H, CH₂OH and —C(O)NHR′, C(O)NR′₂, OR′, CO₂R′, CH₂OR′, NHR′, N(R′)₂, or combinations thereof, wherein R′ is a C₁ to C₃ alkyl or a halogenated alkyl. The substituted heteroalkyl can be substituted with 1 to 3 halogens, a C₁ to C₃ alkyl, OH, NH₂, C(O)NH₂, CO₂H, CH₂OH and —C(O)NHR′, C(O)NR′₂, OR′, CO₂R′, CH₂OR′, NHR′, N(R′)₂, or combinations thereof, wherein R′ is a C₁ to C₃ alkyl or a halogenated alkyl. The substituted heterocycle can be substituted with 1 to 3 halogens, a C₁ to C₃ alkyl, OH, NH₂, C(O)NH₂, CO₂H, CH₂OH and —C(O)NHR′, C(O)NR′₂, OR′, CO₂R′, CH₂OR′, NHR′, N(R′)₂, or combinations thereof, wherein R′ is a C₁ to C₃ alkyl or a halogenated alkyl. In some aspects, R₁ can be hydrogen, a C₁ to C₈ alkyl, a substituted C₁ to C₈ alkyl, a C₃ to C₇ cycloalkyl, or a substituted C₃ to C₇ cycloalkyl. In some aspects, R₂ can be a C₁ to C₈ alkyl, an aryl or a substituted aryl. In some aspects, R₃ and R₄ can be independently hydrogen, a C₁ to C₈ alkyl, a substituted C₁ to C₈ alkyl, a C₃ to C₇ cycloalkyl, or a substituted C₃ to C₇ cycloalkyl. In some aspects, R₅ can be hydrogen, a C₁ to C₈ alkyl, a substituted C₁ to C₈ alkyl, a C₃ to C₇ cycloalkyl, or a substituted C₃ to C₇ cycloalkyl. In some aspects, n is 1. In some aspects, the glucosyl ester can have relatively low crowded space around the carbonyl group of the ester. In some embodiments, one or more glucosyl ester of Formula I can be excluded.

In some particular aspects, X can be CR₃R₄; R₁, R₃, and R₄ can be independently hydrogen, or a C₁ to C₈ alkyl; and R₂ can be an aryl or a substituted aryl. In some particular aspects, X can be CR₃R₄; R₁ is hydrogen or methyl; R₃ and R₄ are independently hydrogen or methyl; and R₂ is a substituted aryl. In some particular aspects, X is CR₃R₄; n is 1; R₁ is methyl, R₃ and R₄ are hydrogen; and R₂ is -pC₆H₄CH₃ and the glucosyl ester can have a chemical formula of Formula II:

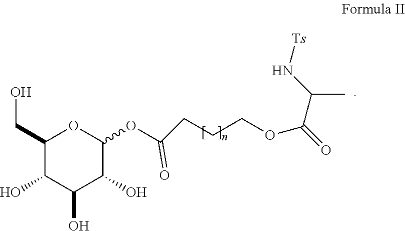

In some aspects, the glucosyl ester can have a structure illustrated in FIG. 1A.

In some particular aspects, X is CR₃R₄; R₁ is methyl, R₃ and R₄ are methyl; and R₂ is -pC₆H₄CH₃ and the glucosyl ester has a chemical formula of Formula III:

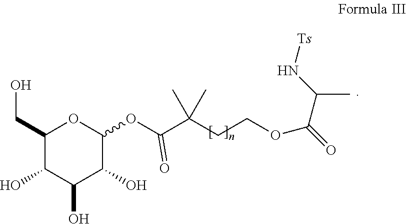

In some aspects, the glucosyl ester can have a structure illustrated in FIG. 1B.

In some particular aspects, the X is NR₅; R₁ and R₅ are independently hydrogen, or a C₁ to C₈ alkyl; and R₂ is an aryl or a substituted aryl. In some particular aspects, the X is NR₅; R₁ and R₅ are independently hydrogen or methyl; and R₂ is a substituted aryl. In some particular aspects, the X is NR₅; R₁ is methyl; R₂ is -pC₆H₄CH₃; and R₅ is hydrogen and the glucosyl ester can have a chemical formula of Formula IV:

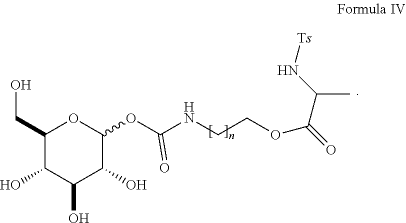

In some aspects, the glucosyl ester can have a structure illustrated in FIG. 1C.

In some particular aspects, the X is NR₅; R₁ is methyl; R₂ is -pC₆H₄CH₃; and R₅ is methyl and the glucosyl ester can have a chemical formula of Formula V:

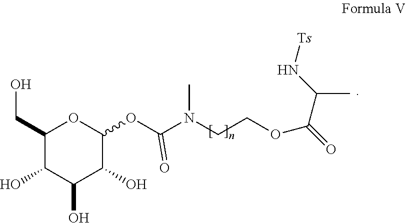

In some aspects, the glucosyl ester can have a structure illustrated in FIG. 1D.

Certain embodiments are directed to a method for detecting leukocyte esterase (LE) activity in a sample. The method includes contacting the sample with a glucosyl ester described herein forming a test sample and determining cleavage of the glucosyl ester by LE in the test sample. In some aspects, the sample can be contacted with an effective amount of the glucosyl ester to form the test sample. The cleavage of the glucosyl ester by LE can be determined by measuring concentration of glucose liberated in the test sample by the cleavage of the glucosyl ester by LE. In some aspects, the initial glucosyl ester concentration in the test sample can be between 1.0, 10, 50, 100, 500, 750 and 1000, 1250, 1500, 1750, 2000 mg/L, including all values and ranges there between. The initial glucosyl ester concentration in the test sample refers to the glucosyl ester concentration in the test sample prior to any enzymatic cleavage of the glucosyl ester by the LE in the test sample. In some aspects, the sample can be contacted with the glucosyl ester (e.g., incubated with the glucosyl ester) for about, at least, or at most 1, 2, 5, 10, 15, 20, 25, 30, 35, 40, 45, 50, 55 min to 1, 1.5, 2, 2.5, 3, 3.5 hour. In certain aspects the incubation time is between 5 min to 3 hours. In some aspects, concentration of the glucose liberated can be measured by determining concentration of glucose in the test sample after contacting the sample with the glucosyl ester to obtain a test glucose concentration, and comparing the test glucose concentration with a control level. The control level can be glucose concentration in the sample before contacting the sample with the glucosyl ester. In some aspects, the glucose concentration can be measured by an electrochemical detection method. In some aspects, the glucose concentration can be measured using an existing glucose detection device and/or method. In some aspects, the glucose concentration can be measured using a glucose strip. The glucose strip can be a glucose strip known in the art. In some particular aspects, the sample can be contacted with a glucosyl ester having a structure illustrated in FIGS. 1A to 1D. In some aspects, one or more glucosyl ester having a structure illustrated in FIGS. 1A to 1D can be excluded.

Certain embodiments are directed to a method for treating an infection (e.g., a microbial infection) or inflammation in a subject. The method can include contacting a sample from the subject with a glucosyl ester described herein forming a test sample, determining glucose concentration in the test sample, and administering a treatment of the infection to the subject if the glucose concentration in the test sample is elevated with respect to a non-infected control. In some aspects, the sample can be contacted with an effective amount of the glucosyl ester to form the test sample. The non-infected control can refer to a glucose concentration in a non-infected state or a reference. In some aspects, the initial glucosyl ester concentration in the test sample can be above 5.0 mg/L. In some aspects, the initial glucosyl ester concentration in the test sample can be between 5.0, 10, 50, 100, 500, 750 and 1000, 1250, 1500, 1750, 2000 mg/L, including all values and ranges there between. The initial glucosyl ester concentration in the test sample refers to the glucosyl ester concentration in the test sample prior to any enzymatic cleavage of the glucosyl ester by the LE in the test sample. In some aspects, the sample can be contacted with the glucosyl ester (e.g., incubated with the glucosyl ester) for about, at least, or at most 1, 2, 5, 10, 15, 20, 25, 30, 35, 40, 45, 50, 55 min to 1, 1.5, 2, 2.5, 3, 3.5 hour. In certain aspects the incubation time is between 5 min to 3 hours. In some aspects, the subject can be human or animal. In some aspects, the sample can be a biological fluid such as blood, plasma, serum, tears, urine, synovial (joint) fluid, or saliva sample. In some aspects, the microbial infection can be a bacterial infection. In some aspects, the administered treatment of the infection can be a known treatment of infection (e.g., antibiotics and the like). In some aspects, administering a treatment of the infection can include administering an effective amount of an antibiotic to the subject. The inflammations marked by the elevated level of white blood cells (increased LE activity): (i) in synovial fluid as a marker of periprosthetic joint infection, (ii) in urine as a marker of urinary tract infection, inflammation of kidneys, prostate cancer, bladder cancer, kidney cancer, and/or (iii) in saliva as a marker of gingival and periodontal diseases. In some aspects, the infection can be a urinary tract infection or periprosthetic joint infection. In certain aspects the assays can be used to detect inflammation of kidneys, prostate cancer, bladder cancer, or kidney cancer. In other aspects, detection of LE in saliva is indicative of gingival or periodontal disease. In some particular aspects, the sample can be contacted with a glucosyl ester having a structure illustrated in FIG. 1A, 1B, 1C, or 1D. In some aspects, one or more glucosyl ester having a structure illustrated in FIG. 1A, 1B, 1C or ID can be excluded.

Certain embodiments are directed to a composition containing a glucosyl ester described herein. Certain embodiments are directed to a kit containing a glucosyl ester described herein. In some aspects, the kit further includes a glucose detection component. The glucose detection component can be glucose detection device (e.g., a glucose strip) and/or reagent. The glucose detection device and/or reagent can be a known glucose detection device and/or reagent. In some particular aspects, the composition can include a glucosyl ester having a structure illustrated in FIG. 1A, 1B, 1C, or 1D. In some particular aspects, the kit can include a glucosyl ester having a structure illustrated in FIG. 1A, 1B, 1C, or 1D. In some aspects, one or more glucosyl ester having a structure illustrated in FIG. 1A, 1B, 1C, or 1D can be excluded.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

“Measuring leukocyte esterase,” as used herein, is detecting the presence of leukocyte esterase; or quantitatively or semi-quantitatively measuring the amount or concentration of leukocyte esterase in a sample. Systems, kits, device, and/or reagents that can be used for measuring leukocyte esterase are described more fully herein. “Determining concentration of glucose” as used herein, is detecting the presence of glucose; or quantitatively or semi-quantitatively measuring the amount or concentration of glucose in a sample. Systems, kits, device, and/or reagents that can be used for measuring glucose are described more fully herein.

The term “effective,” as that term is used in the specification and/or claims, means adequate to accomplish a desired, expected, or intended result.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “comprise”, “have”, and “include” are open-ended linking verbs. Any forms or tenses of one or more of these verbs, such as “comprises”, “comprising”, “has”, “having”, “includes”, and “including”, are also open-ended. For example, any method that “comprises”, “has”, or “includes”, one or more steps is not limited to possessing only those one or more steps and also covers other unlisted steps.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present invention can “comprise,” “consist essentially of,” or “consist of” particular ingredients, components, blends, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1D. Examples of a compounds of Formula I. (FIG. 1A) (2S,3R,4S,5S,6R)-3,4,5-trihydroxy-6-(hydroxymethyl)oxan-2-yl 4-{[(2S)-2-(4-methylbenzenesulfonamido)propanoyl]oxy}butanoate (CIDD-0150156). (FIG. 1B) (2S,3R,4S,5S,6R)-3,4,5-trihydroxy-6-(hydroxymethyl)oxan-2-yl 2,2-dimethyl-4-{[(2S)-2-(4-methylbenzenesulfonamido)propanoyl]oxy}butanoate (CIDD-0150181). (FIG. 1C) 2-[({[(2R,3R,4S,5S,6R)-3,4,5-trihydroxy-6-(hydroxymethyl)oxan-2-yl]oxy}carbonyl)amino]ethyl (2S)-2-(4-methylbenzenesulfonamido)propanoate (CIDD-0150161). (FIG. 1D) 2-[methyl({[(2R,3R,4S,5S,6R)-3,4,5-trihydroxy-6-(hydroxymethyl)oxan-2-yl]oxy}carbonyl)amino]ethyl (2S)-2-(4-methylbenzenesulfonamido)propanoate (CIDD-0150157).

FIG. 2. The Accu-Check glucose test strip inserted in a DropSens electronic holder ready to be contacted with a small (˜10 μL) drop of analyzed bodily fluid placed on a Petri dish.

FIGS. 3A-3B. (FIG. 3A) The charge Q flowing through a commercial glucose test strip that was contacted at time t=0 s with a pH 7.40 PBS solution containing (a) 0, (b) 1.0, (c) 10, (d) 20, (e) 50, (f) 75, and (g) 100 μM glucose. The Q values were obtained by integrating amperograms (0.15 V), which were recorded with a laboratory potentiostat, between t=2 and 22 s. Potential, 0.15 V vs. strip's counter/reference electrode. (FIG. 3B) The calibration plot based on data in panel A. The ΔQ=Q_{(glucose)}−Q_{(no glucose)}, where Q_{(glucose)} and Q_{(no glucose)} were the charges measured in the presence and absence of glucose in a solution.

FIG. 4. Dependence of signal ΔQ vs. incubation time for a pH 7.40 PBS solution containing both 25 μg L⁻¹ LE and 5.5 mM ester α. The ΔQ=Q₍ₑₛₜₑᵣ₎−Q_{(no ester)}, where Q₍ₑₛₜₑᵣ₎ and Q_{(no ester)} are the charges flowing through a glucose strip in contact with an ester-incubated solution and ester-free solution, respectively. Potential, 0.15 V vs. glucose strip's counter/reference electrode.

FIGS. 5A-5B. The charge Q flowing through a commercial glucose test strip that was contacted at time t=0 s with human (FIG. 5A) synovial fluid containing (a) 0, (b) 50, (c) 110, (d) 250 and (e) 800 μg L⁻¹ LE, and (FIG. 5B) urine containing (a) 0, (b) 25, (c) 80, (d) 150 and (e) 500 μg L⁻¹ LE. The Q values were obtained by integrating amperograms (0.15 V), which were recorded with a laboratory potentiostat, between t=2 and 22 s. The synovial fluid and urine samples were mixed with a stock solution of ester α (5% sample dilution; final ester concentration, 5.5 mM) and incubated for 5 min before contacting them with a glucose strip. Potential, 0.15 V vs. strip's reference/counter electrode.

FIG. 6. Dependence of signal ΔQ on LE concentration in human synovial fluid (red circles) and urine (black squares) samples. The samples were incubated with 5.5 mM ester α for 5 min. The ΔQ=Q₍ₑₛₜₑᵣ₎−Q_{(no ester)}, where Q₍ₑₛₜₑᵣ₎ and Q_{(no ester)} are the charges flowing through a glucose strip in contact with an ester-incubated solution and ester-free solution, respectively. The Q values were obtained by integrating amperograms (0.15 V), which were recorded with a laboratory potentiostat, between t=2 and 22 s. The error bars (±SD) for points at <110 μg L⁻¹ LE were smaller than markers. The different intensities of purple show a response of commercial colorimetric LE test strips to increasing concentration of LE (from trace to +, ++, +++).

FIGS. 7A-7B. (FIG. 7A) The charge Q flowing through a commercial glucose test strip that was contacted at time t=0 s with the glucose-loaded (18 mM) urine containing (a) 0, (b) 25, (c) 80, (d) 150 and (e) 500 μg L⁻¹ LE. The Q values were obtained by integrating amperograms (0.15 V), which were recorded with a laboratory potentiostat, between t=2 and 22 s. The urine samples were mixed with a stock solution of ester α (5% sample dilution; final ester concentration, 5.5 mM) and incubated for 5 min before contacting them with a glucose strip. (FIG. 7B) Dependence of signal ΔQ on LE concentration in urine samples that contained glucose at 0 (black squares) and 18 mM (white squares, data from panel A). The different intensities of purple show a response of commercial colorimetric LE test strips to increasing concentration of LE (from trace to +, ++, +++).

FIG. 8. Dependence of signal ΔQ on LE concentration in human urine that was incubated for 5 min with ester α (black squares) or 10 min with ester γ (red triangles). The ΔQ=Q₍ₑₛₜₑᵣ₎−Q_{(no ester)}, where Q₍ₑₛₜₑᵣ₎ and Q_{(no ester)} are the charges flowing through a glucose strip in contact with an ester-incubated solution and ester-free solution, respectively. The amperograms (0.15 V) were recorded with a laboratory potentiostat and integrated between t=2 and t=22 s to obtain Q values. The different intensities of purple show a response of commercial colorimetric LE test strips to increasing concentration of LE (from trace to +, ++, +++).

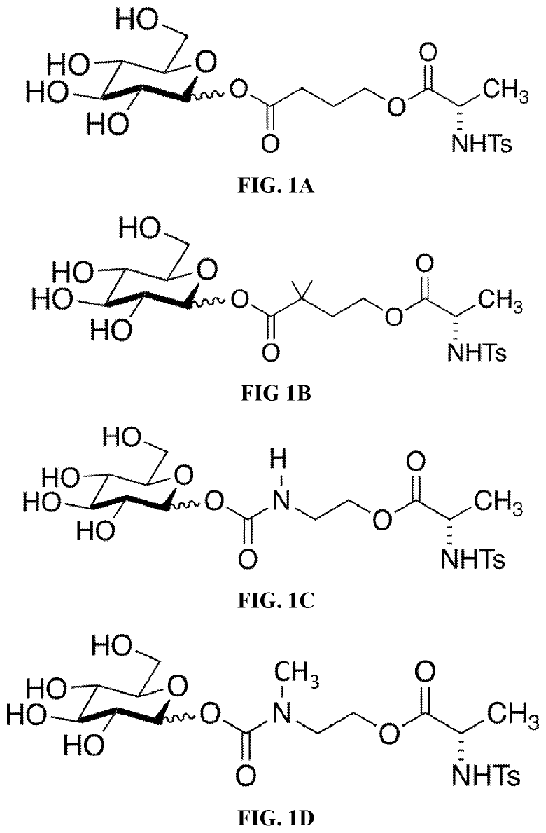

### DETAILED DESCRIPTION OF THE INVENTION

Embodiments of the present invention include compounds, devices, and/or diagnostic methods for detecting or evaluating the presence of infection or inflammation in humans or animals. These methods can include measuring the amount of leukocyte esterase (LE) present in a sample obtained from the human or animal being tested or diagnosed. Amount of leukocyte esterase (LE) present in the sample can be measured by contacting the sample with a glucosyl ester described herein to obtain a test sample and measuring concentration of glucose liberated in the test sample by cleavage of the glucosyl ester by LE. Certain embodiments of the present invention provides a relatively high-resolution quantification of a degree of infection in a biological fluid irrespective of their state (opacity, color). The biological fluid can be blood, plasma, serum, tears, urine, synovial (joint) fluid or saliva or other bodily fluids and effusions for infections. The glucosyl esters described herein are substrates for LE and release glucose in the presence of active enzyme leukocyte esterase (LE), which is a proxy for the presence of leukocytes and the marker of common infections. Synthesis of such esters, including their structural requirements, and the coupling of their enzymatic reactions to commercial glucose test strips is described herein.

### A. Glucosyl Esters

Certain embodiments are directed to glucosyl ester of Formula I as substrates for LE. In certain aspects the glucosyl ester can be utilized in an assay of enzymatic activity of leukocyte esterase (LE) for the rapid and accurate diagnosis of the presence and extent of infection in human and animal samples.

Compounds of Formula I or their salts such as pharmaceutically acceptable salts or solvates thereof, can be prepared according to reaction Scheme 1, 2, and 3 below. Isolation and purification of the products is accomplished by standard procedures, which are known to a chemist of ordinary skill. The following schemes and examples provide examples of the processes for making compounds of Formula I. It is to be understood, however, that the invention, as fully described herein and as recited in the claims, is not intended to be limited by the details of the following examples. Unless otherwise noted, n and R₁ through R₅ are defined as above.

Referring to scheme 1, a compound of the formula 1 is reacted with chloroacetyl chloride in the presence of a base such as pyridine in a solvent such as dichloromethane at a temperature from 0° C. to room temperature for a time from 1 hour to 24 hours. The resulting compound, 2, is treated with hydrazine acetate in a solvent such as dimethylformamide at 0° C. for a time from 1 hour to 2 hours. The resulting compound, 3, is coupled with reactive components such as trichloroacetonitrile or 4-nitrophenyl chloroformate in the presence of base such as 1,8-diazabicyclo[5.4.0]undec-7-ene or triethylamine in a solvent such as dichloromethane, at a temperature from 0° C. to room temperature for a time from 1 hour to 24 hours. The resulting glycosyl donors 4 and 5 are reacted with the glycosyl acceptors such as compounds 9 and 12 as described in Scheme 2.

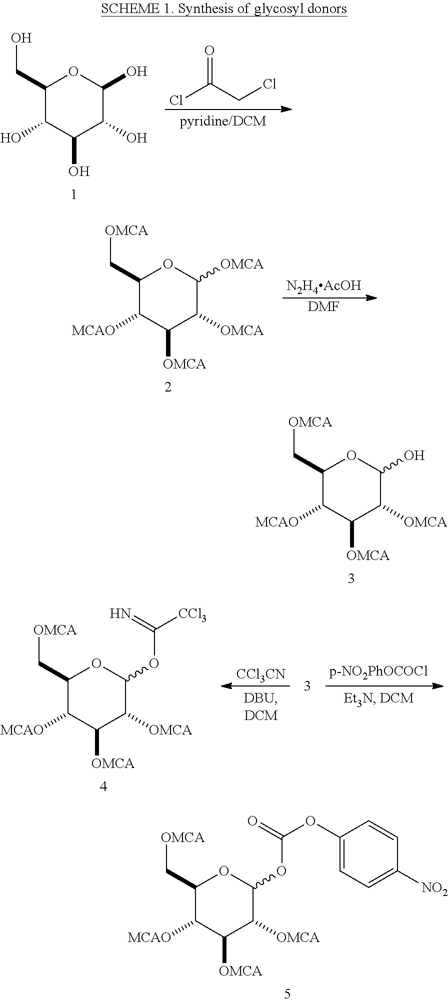

Referring to Scheme 2, a compound of the formula 6 is reacted with substituted hydroxyl reagent such as compound 7 or 10 in the presence of a carboxylic acid activating reagent such as EDCI and DMAP in a solvent such as dichloromethane at a temperature from 0° C. to room temperature for a time from 1 hour to 24 hours. The resulting compound, 8 and 11, is individually treated with an organic/inorganic acid, such as TFA or hydrochloric acid, in a solvent such as ether, dioxane, or dichloromethane, at a temperature from 0° C. to room temperature for a time from 1 hour to 24 hours. In some aspects, R₁ can be H or a C₁ to C₈ alkyl. In some particular aspects, R₁ can be methyl. In some aspects, R₂ can be aryl or substitutes aryl. In some particular aspects, R₂ can be pC₆H₄CH₃. In some aspects, R₂ and R₃ can be independently H or a C₁ to C₈ alkyl. In some particular aspects, R₂ and R₃ can be independently H or methyl. In some particular aspects, R₂ and R₃ can be H or methyl. In some aspects, R₅ can be H or a C₁ to C₈ alkyl. In some particular aspects, R₅ can be H or methyl. In some particular aspects, n can be 1. The resulting compounds, 9 and 12, are reacted with the glycosyl donors such as compounds 4 and 5 as described in Scheme 1.

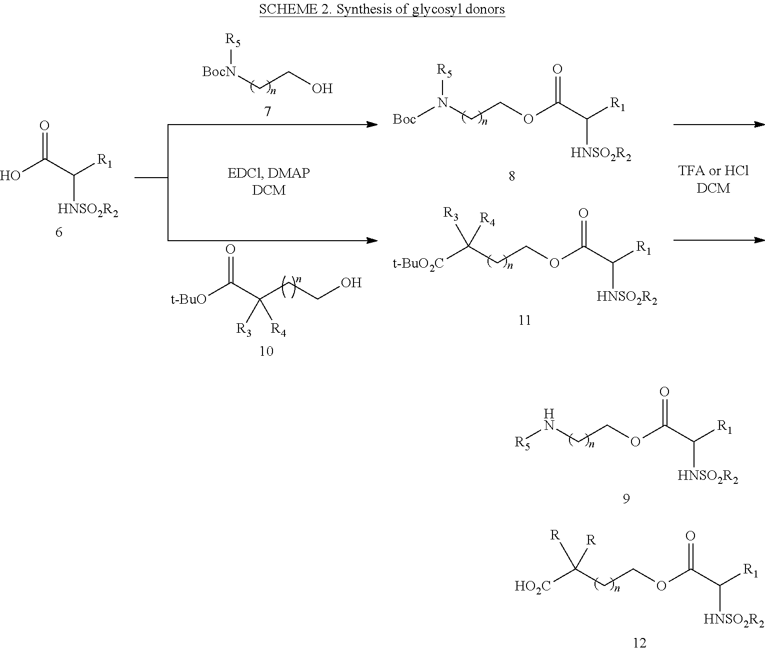

Referring to Scheme 3, a compound of the formula 5 is reacted with a glycosyl acceptor such as compounds 9 in the presence of an organic base such as DIPEA or triethylamine in a solvent such as dichloromethane at a temperature from 0° C. to room temperature for a time from 1 hour to 24 hours to form a compound having the chemical formula of Formula I where X is NR₅. Alternatively, a compound of the formula 4 is reacted with a glycosyl acceptor such as compounds 12 in the presence of an organic acid such as TMSOTf and molecular sieves in a solvent such as dichloromethane at a temperature from 0° C. to room temperature for a time from 1 hour to 24 hours to form a compound having the chemical formula of Formula I where X is CR₃R₄. Finally, removal of the MCA protecting groups is accomplished in the presence of a tertiary amine or an organic amine base such as DIPEA or pyridine in a solvent such as water at a temperature from 0° C. to room temperature for a time from 1 hour to 24 hours.

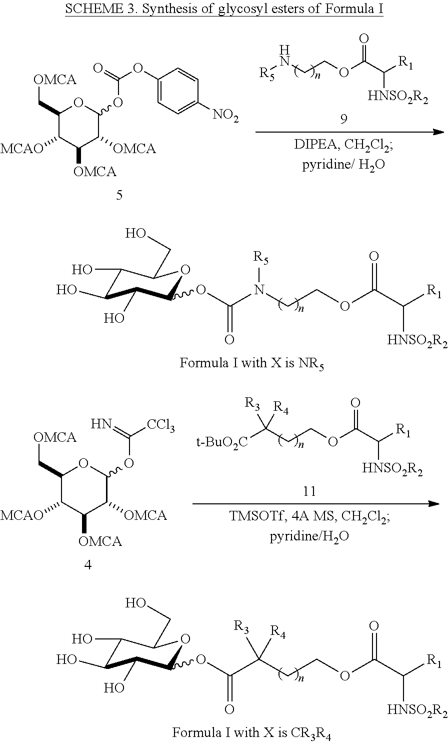

Finally, salts such as pharmaceutically acceptable salts of compounds of Formula I may be prepared by one or more of three methods: (i) by reacting the compound of Formula I with the desired acid; (ii) by removing an acid-labile protecting group from a suitable precursor of the compound of Formula I or by ring-opening a suitable cyclic precursor, for example, a lactone or lactam, using the desired acid or base; or (iii) by converting one salt of the compound of Formula I to another by reaction with an appropriate acid or base or by means of a suitable ion exchange column.

Pharmaceutically acceptable salts of the compounds of Formula I include the acid or base addition salts thereof. All three reactions are typically carried out in solution. The resulting salt may precipitate out and be collected by filtration or may be recovered by evaporation of the solvent. The degree of ionization in the resulting salt may vary from completely ionized to almost non-ionized. Suitable non-toxic, acid-addition pharmaceutically acceptable salts include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable non-toxic, base-addition pharmaceutically acceptable salts include, but are not limited to aluminium, arginine, benzathine, calcium, choline, diethylamine, diolamine, glycine, lysine, magnesium, meglumine, olamine, potassium, sodium, tromethamine and zinc salts. For a review on suitable salts, see Handbook of Pharmaceutical Salts: Properties, Selection, and Use by Stahl and Wermuth (Wiley-VCH, 2002).

Included within the scope of the present invention are all stereoisomers, geometric isomers and tautomeric forms of the compounds of Formula I, including compounds exhibiting more than one type of isomerism, and mixtures of one or more thereof.

The present invention can include all pharmaceutically acceptable isotopically-labelled compounds of Formula I wherein one or more atoms are replaced by atoms having the same atomic number, but an atomic mass or mass number different from the atomic mass or mass number which predominates in nature.

### B. Chemical Definitions

Various chemical definitions related to such compounds are provided as follows.

As used herein, “predominantly one enantiomer” means that the compound contains at least 85% of one enantiomer, or more preferably at least 90% of one enantiomer, or even more preferably at least 95% of one enantiomer, or most preferably at least 99% of one enantiomer. Similarly, the phrase “substantially free from other optical isomers” means that the composition contains at most 5% of another enantiomer or diastereomer, more preferably 2% of another enantiomer or diastereomer, and most preferably 1% of another enantiomer or diastereomer.

As used herein, the term “water soluble” or “hydrophilic” means that the compound dissolves in water. In the context of a LE assay, “water soluble” is the minimum concentration of LE substrate that generates a measurable amount of current from the LE+substrate reaction, which can be as low as 1 micromole/liter.

As used herein, the term “nitro” means —NO₂; the term “halo” or “halogenated” designates —F, —Cl, —Br or —I; the term “mercapto” means —SH; the term “cyano” means —CN; the term “azido” means —N₃; the term “silyl” means —SiH₃, and the term “hydroxyl” means —OH.

The term “alkyl,” by itself or as part of another substituent, means, unless otherwise stated, a linear (i.e. unbranched) or branched carbon chain, which may be fully saturated, mono- or polyunsaturated. An unsaturated alkyl group is one having one or more double bonds or triple bonds. Saturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂(iso-Pr), —CH₂CH₂CH₂CH₃ (n-Bu), —CH(CH₃)CH₂CH₃ (sec-butyl), —CH₂CH(CH₃)₂(iso-butyl), —C(CH₃)₃(tert-butyl), —CH₂C(CH₃)₃ (neo-pentyl), are all non-limiting examples of alkyl groups. C₁ alkyl refers to alkyl group with n carbon atoms, e.g. C₁ refers to methyl.

The term “halogenated alkyl” means a straight-chain or branched saturated monovalent hydrocarbon group of one to twelve carbon atoms, wherein at least one of the carbon atoms is replaced by a halogen atom (e.g. fluoromethyl, 1-bromo-ethyl, 2-chloro-pentyl, 6-iodo-hexyl, and the like).

The term “heteroalkyl” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of 0 and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The terms “cycloalkyl” and “heterocycle,” by themselves or in combination with other terms, means cyclic versions of “alkyl” and “heteroalkyl”, respectively. Additionally, for heterocycle, a heteroatom can occupy the position at which the heterocycle is attached to the remainder of the molecule. C₁ cycloalkyl refers to cycloalkyl group with n carbon atoms, e.g C₃ refers to cyclopentyl.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl). In certain aspects a heterocycle or a heteroaryl is optionally substituted with 1 to 3 halogens, C₁ to C₃ alkyl, OH, NH₂, C(O)NH₂, CO₂H, CH₂OH and —C(O)NHR′, C(O)NR′₂, OR′, CH₂OR′, NHR′ or N(R′)₂, where in R′ is C₁ to C₃ alkyl or halogenated alkyl. In other aspects a cycloakyl can be optionally substituted with 1 to 3 halogens, C₁ to C₃ alkyl, OH, NH₂, C(O)NH₂, CO₂H, CH₂OH, C(O)NHR″, C(O)NR″₂, OR″ CH₂OR″, NHR″, —N(R″)₂ or combinations thereof, where R″ is C₁ to C₃ alkyl or halogenated alkyl.

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)—R′, where R′ is an alkyl group. R′ may have a specified number of carbons (e.g. “C₁₋₄ alkylsulfonyl”).

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use by Stahl and Wermuth (Wiley-VCH, 2002) which is incorporated herein by reference.

An “isomer” of a first compound is a separate compound in which each molecule contains the same constituent atoms as the first compound, but where the configuration of those atoms in three dimensions differs. Unless otherwise specified, the compounds described herein are meant to encompass their isomers as well. A “stereoisomer” is an isomer in which the same atoms are bonded to the same other atoms, but where the configuration of those atoms in three dimensions differs. “Enantiomers” are stereoisomers that are mirror images of each other, like left and right hands. “Diastereomers” are stereoisomers that are not enantiomers.

### C. Leukocyte Esterase Assay

The glucosyl esters described herein can function as substrates for LE, and can get cleaved by LE to release glucose, reaction (1). Amount of glucose released can be proportional to the LE activity:

$$glucosyl ester {\to }^{LE} glucose \qquad ( 1 )$$

In some aspects, LE activity in a sample can be measured by contacting the sample with a glucosyl ester described herein to form a test sample and measuring concentration of glucose released in the test sample by cleavage of the glucosyl ester by LE. The glucose released can be detected with a glucose detecting component. In some aspects, concentration of the glucose released can be measured by a glucose test strip, by measuring electrical charge flowing through the glucose test strip. In some aspects, the test sample can be contacted with a glucose test strip, such as a known glucose test strip, and charge flowing through the glucose test strip can be measured to obtain glucose concentration in the test sample. In some aspects, the charge flowing through the glucose test strip can be measured with a potentiostat or a glucometer.

Glucosyl ester described herein can be incorporated into diagnostic products or kits. The diagnostic products can be used for detecting LE activity by detecting glucose. In certain aspects, the diagnostic products can include at least one compound or agent useful in detecting the presence and/or concentration of glucose. The term “compound or agent useful in detecting the presence of glucose”, as used herein, refers to a compound, composition, or combination thereof that is changed by presence and/or concentration of glucose.

Diagnostic kits can be useful for detecting LE activity by detecting glucose. In certain aspects kits can include a device or apparatus or product for collecting a sample such as a biological fluid from a human or an animal being tested or diagnosed, and assay or assay device for measuring the amount of glucose released in the sample after the sample is contacted with a glucosyl ester described herein.

The phrase “device or apparatus for collecting a sample”, as used herein, means any device or apparatus or product which is useful for removing a sample of fluid, tissue, or cells from a human or animal being tested or diagnosed without adversely affecting the ability to detect the presence of leukocyte esterase activity in the sample. Non-limiting examples of such devices include swabs, pipettes, syringes, absorbent tapes, absorbent gauzes, absorbent strips, scoops, suction bulbs, and aspirators. A kit can include one or more diagnostic products described herein.

In certain aspects the kits can be manufactured such that the sample collecting device and the assay device are separate components in the kits. The kit can include optional components to be used with the kits (e.g. test tubes for diluting samples in; bottles containing dilution fluid for diluting samples; instruction sheets; etc.) that can be combined into one package. An example of such a package is a box which is shrink wrapped with plastic.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Glucosyl Ester Substrates for Enzyme Leukocyte Esterase (LE)

Reagents and Solutions. The esters a (CIDD-0150156, MW: 491.51 g mol⁻¹), R (CIDD-0150181, MW: 506.52 g mol⁻¹), γ (CIDD-0150161, MW: 492.5 g mol⁻¹), and κ (CIDD-0150157, MW:519.56 g mol⁻¹) were synthesized in the Center for Innovative Drug Discovery (CIDD) at the University of Texas at San Antonio.

The human leukocyte suspension in 154 mM NaCl solution (cat. No. MBS173116, 0.0867 mg mL⁻¹ leukocyte esterase (LE) protein, 4×10⁸ WBC mL⁻¹) was purchased from MyBioSource (San Diego, Calif.). The suspension was diluted 10 times with a 90:10 vol. % mixture of 50 mM phosphate buffer saline (PBS, pH 7.40, 154 mM NaCl) solution and dimethylsulfoxide, left for 10 min to lyse the leukocytes chemically, sonicated for 30 s with a Q125 Qsonica probe (20% power) to further lyse them mechanically, and centrifuged at 15 000×g for 30 min. The resulting supernatant that contained LE was stored at −20° C.

The human synovial fluid (DLS0092658) was purchased from Discovery Life Sciences (Los Osos, Calif.) and stored at −70° C. when not in use. The clean-catch midstream portion of human urine (˜15.0 mL) was collected early in the morning in a 50.0-mL sterile centrifuge tube (Fisher Scientific, Pittsburgh, Pa.) centrifuged at 600×g for 5 min, and the supernatant was stored at −20° C. The synovial fluid and urine were spiked with known amount of LE before the analysis.

The glucose test strips (ACCU-CHECK Aviva Plus, Roche Diabetes Care Inc., Indianapolis, Ind.) and colorimetric urinary infection LE test strips (Siemens Multistix 10 SG) were purchased locally. The correlation between the color zones of colorimetric LE strips (trace, +, ++, +++) and the concentration of LE was established by using a PBS solution, which was spiked with a known amount of LE (μg L⁻¹).

Electrochemical Measurements. The glucose strip was inserted into an electronic holder (DropSens, Llanera, Spain), which was then connected to a CHE 832B potentiostat (other potentiostats could also be used). The strip was used in a 2-electrode mode with the counter and reference electrodes connected together as a single cathode and a working electrode serving as an anode (the strip's fill-detector electrode was not used). The potential applied to a working electrode was equal to 0.15 V vs. strip's reference/counter electrode.

The 0.15 V potential was applied to a glucose strip, which was then contacted at time t=0 s with a small drop of sample (˜10 μL) that was placed on a Petri dish (FIG. 2). The amperogram obtained in such a way was integrated between t=2 and t=22 s to determine the charge Q flowing through the strip. Two charges were always determined: Q₍ₑₛₜₑᵣ₎ for a sample incubated with 5.5 mM ester for 5 min, and Q_{(no ester)} for an original sample without ester. The difference in charge ΔQ [=Q₍ₑₛₜₑᵣ₎−Q_{(no ester)}] was proportional to the enzymatic activity of LE in a sample.

General procedures. All operations were carried out at room or ambient temperature, that is, in the range of 18-25° C.; evaporation of solvent was carried out using a rotary evaporator under reduced pressure with a bath of up to 60° C.; reactions were monitored by thin layer chromatography (tlc) and reaction times are given for illustration only; melting points (m.p.) given are uncorrected (polymorphism may result in different melting points); structure and purity of all isolated compounds were assured by at least one of the following techniques: tlc (Merck silica gel 60 F-254 precoated plates), high performance liquid chromatography (HPLC), mass spectrometry, nuclear magnetic resonance (NMR) or infrared spectroscopy (IR). Yields are given for illustrative purposes only. Flash column chromatography was carried out using Merck silica gel 60 (230-400 mesh ASTM). Low-resolution mass spectral data (EI) were obtained on platform 1: an Agilent 1290 series HPLC system (Method 1) comprised of binary pumps, degasser and UV detector, equipped with an auto-sampler that is coupled with Agilent 6150 mass spectrometer; platform 2: a Thermo Scientific Vanquish UHPLC system. The general Liquid Chromatography parameters were as follows using solvent A (0.10% formic acid in water) and solvent B (0.00% formic acid in acetonitrile): Method 1: analysis was performed on a Zorbax Eclipse Plus C18 column with dimension of 2.1×50 mm. The flow rate was 0.7 ml/minute running a gradient of 5% to 95% solvent B in 5 minutes and hold at 95% solvent B for 2 minutes. Method 2: analysis was performed on a Hypersil GOLD C18 column with dimension of 2.1×100 mm. The flow rate was 1.0 ml/minute running a gradient of 5% to 95% solvent B in 0.8 minutes and hold at 95% solvent B for 0.4 minutes. The ionization type for the mass detector of the mass spectrophotometer was atmospheric pressure electrospray in the positive ion mode with a fragmentor voltage of 50 volts. NMR data was determined at 400 MHz (Agilent DD2 400 MHz spectrometer) using deuterated chloroform (99.8% D), methanol (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, m=multiplet, br=broad, etc.

The following abbreviations are used:

K₂CO₃: potassium carbonate; MeCN: acetonitrile; NaOH: sodium hydroxide; MeOH: methanol; SOCl₂: thionyl chloride; DMF: dimethylformamide; CH₂Cl₂: dichloromethane; THF: tetrahydrofuran; Et₃N: triethylamine; Pd/C: palladium on activated carbon; EtOH: ethanol; NaHCO₃: sodium bicarbonate; HCl: hydrogen chloride; EtOAc: ethyl acetate; Na₂SO₄: sodium sulfate; MeCN: acetonitrile; MCA: methylchloroacetate; DBU: 1,8-diazabicyclo[5.4.0]undec-7-ene; EDCI: N-(3-Dimethylaminopropyl)-N′-ethylcarbodiimide hydrochloride; DMAP: 4-(Dimethylamino)pyridine; TFA: Trifluoroacetic acid; DIPEA: N,N-Diisopropylethylamine; TMSOTf: Trimethylsilyl trifluoromethanesulfonate.

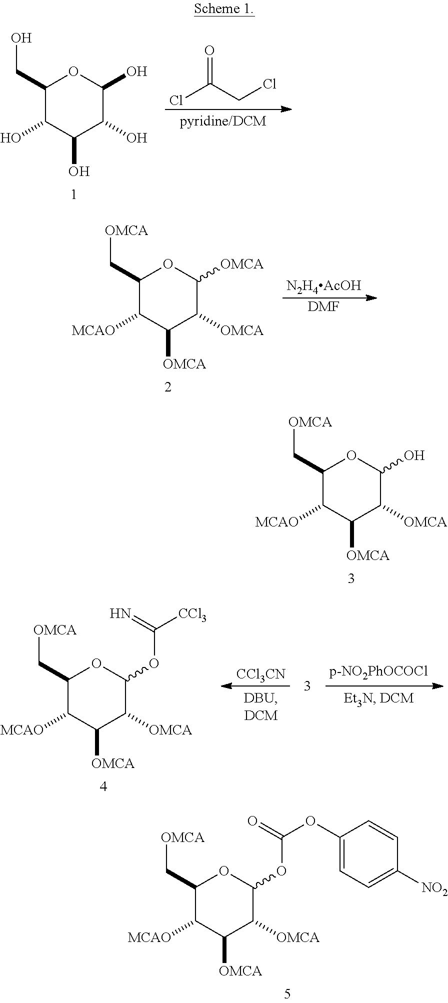

Referring to Scheme 1 above, a compound of the formula 1 is reacted with chloroacetyl chloride in the presence of a base such as pyridine in a solvent such as dichloromethane at a temperature from 0° C. to room temperature for a time from 1 hour to 24 hours. The resulting compound, 2, is treated with hydrazine acetate in a solvent such as dimethylformamide at 0° C. for a time from 1 hour to 2 hours. The resulting compound, 3, is coupled with reactive components such as trichloroacetonitrile or 4-nitrophenyl chloroformate in the presence of base such as 1,8-diazabicyclo[5.4.0]undec-7-ene or triethylamine in a solvent such as dichloromethane, at a temperature from 0° C. to room temperature for a time from 1 hour to 24 hours. The resulting glycosyl donors 4 and 5 are reacted with the glycosyl acceptors such as compounds 9 and 12 as described in Scheme 2.

Oenta(chloroacetyl)glucose 2: A solution of β-D-glucose (5.0 g, 27.7 mmol, 1 equiv) in dry CH₂Cl₂ (110 ml) and dry pyridine (14 ml) at 0° C. was added a solution of chloroacetyl chloride (31.3 g, 277 mol, 10 equiv) in dry CH₂Cl₂ (45 ml) through an addition funnel over a period of 2 hours. The resulting slurry turned from bright orange/red colors to yellowish homogeneous solution after stirring at ambient temperate for 24 hours. The reaction mixture was quenched with 1N HCl (aq.) (100 ml) and the mixture was extracted with CH₂Cl₂ (2×50 ml). The combined organic extracts were washed with saturated NaHCO₃ (aq.) (100 ml), brine (100 ml) and were drived over Na₂SO₄, filtered and concentrated under reduced pressure. The crude material was purified by Biotage flash chromatography (gradient elution, 0 to 35% EtOAc in hexanes) to obtain the title compound 2 (15.2 g, 98%) as viscous yellow oil. ¹H NMR matched the one reported by Y. Zhu, J. Ralph, Tetrahedron Letters, 2011, 52, 3729-3731.

2,3,4,6-tetra-O-chloroacetylglucose 3: A solution of β-D-glucose pentamethylchloroacetate (2) (1.3 g, 2.31 mmol, 1 equiv) in DMF (4 ml) at 0° C. was added hydrazine acetate (255 mg, 2.77 mmol, 1.2 equiv). The reaction mixture was stirred at this temperature for 2 hours. Upon reaction completion based on TLC analysis (50% EtOAc in hexanes), the reaction was diluted with EtOAc (20 ml) and washed with H₂O (20 ml), and the organic extracts were washed with brine and dried over Na₂SO₄, filtered and concentrated under reduced pressure. The crude material was purified by Biotage flash chromatography (gradient elution, 0 to 35% EtOAc in hexanes) to obtain the title compound 3 (883 mg, 79%) as colorless foams, ¹H NMR matched the one reported by Y. Zhu, J. Ralph, Tetrahedron Letters, 2011, 52, 3729-3731.

2,3,4,6-tetra-O-chloroacetylglucosyl trichloroacetimidate 4: A solution of the 2,3,4,6-tetra-O-chloroacetylglucose (3) (1.82 g, 3.74 mmol, 1 equiv) in dry CH₂Cl₂ (12 ml) at 0° C. was added trichloroacetonitrile (5.4 g, 37.4 mmol, 10 equiv) and catalytic amount of DBU (85 mg, 0.561 mmol, 0.15 equiv). The reaction was left stirring at this temperature and gradually raised to ambient temperature for overnight. The reaction mixture was concentrated and the resulting residue was purified by Biotage flash chromatography (gradient elution, 0 to 20% EtOAc in hexanes) to obtain the title compound 4 (2.06 g, 88%) as yellow viscous oil, which was used as is.

2,3,4,6-tetra-O-chloroacetylglucosyl 4-nitrophenylcarbonate 5: A solution of 2,3,4,6-tetra-O-chloroacetylglucose (3) (1.1 g, 2.26 mmol, 1 equiv) in dry CH₂Cl₂ (12 ml) at 0° C. was added triethylamine (343 mg, 3.39 mmol, 1.5 equiv) and 4-nitrophenyl chloroformate (547 mg, 2.71 mmol, 1.2 equiv). The reaction was raised to ambient temperature and stirred for 1 hour. The reaction mixture was concentrated and the resulting residue was purified by Biotage flash chromatography (gradient elution, 0 to 35% EtOAc in hexanes) to obtain the title compound 5 (1.2 g, 81%) as yellow viscous oil, which was used as is.

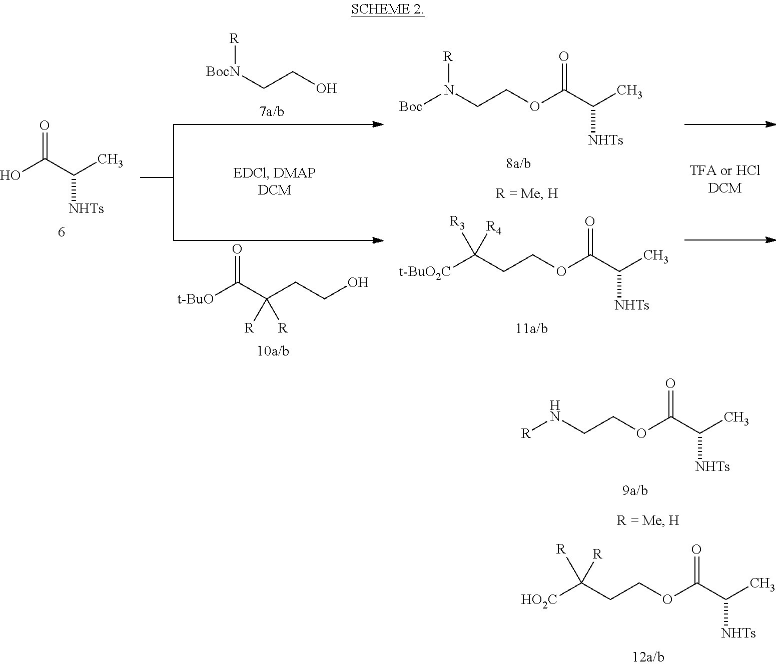

Referring to Scheme 2 above, a compound of the formula 6 is reacted with substituted hydroxyl reagent such as compound 7a/b or 10a/b in the presence of a carboxylic acid activating reagent such as EDCI and DMAP in a solvent such as dichloromethane at a temperature from 0° C. to room temperature for a time from 1 hour to 24 hours. R depicted in scheme 2 is individually selected from groups such as hydrogen, methyl and other alkyl substituents. The resulting compound, 8a/b and 11a/b, is individually treated with an organic/inorganic acid, such as TFA or hydrochloric acid, in a solvent such as ether, dioxane, or dichloromethane, at a temperature from 0° C. to room temperature for a time from 1 hour to 24 hours. The resulting compounds, 9a/b and 12a/b, are reacted with the glycosyl donors such as compounds 4 and 5 as described in Scheme 1.

2-((tert-butoxycarbonyl)(methyl)amino)ethyl tosyl-L-alaninate 8a: A solution of 6 (1 g, 5.70 mmol, 1 equiv) and N-methyl-N-boc-glycinol 7a (R=methyl) (1.45 g, 5.99 mmol, 1.05 equiv) in dry CH₂Cl₂ (15 ml) was added DMAP (350 mg, 2.86 mmol, 0.5 equiv) and EDCI (1.20 g, 6.27 mmol, 1.1 equiv), and the resulting reaction was stirred at room temperature for 24 hours. The reaction was concentrated and purified by Biotage flash chromatography (gradient elution, 0 to 40% EtOAc in hexanes) to obtain the title compound 8a (R=methyl) (1.40 g, 58%) as colorless oil. ¹H NMR (400 MHz, Chloroform-d) δ 7.73 (d, J=8.3 Hz, 2H), 7.29 (d, J=8.2 Hz, 2H), 4.04 (m, 2H), 3.97 (pent, J=7.8 Hz, 1H), 3.44-3.29 (br, 3H). 2.83 (s, 3H), 2.42 (s, 3H), 1.45 (s, 9H), 1.39 (d, J=7.2 Hz, 3H). LCMS (m/z) 301.1 (M-Boc); RT (Method 2, std LCMS method), 1.08 min.

Compound 8b (R=hydrogen) was prepared using the reaction condition described above with the corresponding N-boc-glycinol 7b (R=hydrogen) to give the title compound 8b (R=hydrogen) (1.61 g, 64%) as colorless oil. ¹H NMR (400 MHz, Chloroform-d) δ 7.74 (d, J=8.2 Hz, 2H), 7.30 (d, J=8.2 Hz, 2H), 5.21 (m, 1H), 4.69 (br, 1H), 4.04-3.95 (m, 3H), 3.27 (m, 2H), 2.42 (s, 3H), 1.45 (s, 9H), 1.38 (d, J=7.2 Hz, 3H). LCMS (m/z) 287.2 (M-Boc); RT (Method 2, std LCMS method), 1.25 min.

Compound 11a (R=methyl) was prepared using the reaction condition described above with the corresponding tert-butyl-4-hydroxy-2,2-dimethylbutanoate 10a (R=methyl) to give the title compound 11a (R=methyl) (1.11 g, 68%) as colorless oil. ¹H NMR (400 MHz, Chloroform-d) δ 7.72 (d, J=8.2 Hz, 2H), 7.29 (d, J=8.2 Hz, 2H), 5.18 (m, 1H), 3.95 (m, 3H), 2.41 (s, 3H), 1.70 (m, 2H), 1.43 (s, 9H), 1.37 (d, J=7.1 Hz, 3H), 1.11 (s, 6H).

Compound 11b (R=hydrogen) was prepared using the reaction condition described above with the corresponding tert-butyl 4-hydroxybutanoate 10b (R=hydrogen) to give the title compound 11b (R=hydrogen) (1.96 g, 91%) as colorless oil. ¹H NMR (400 MHz, Chloroform-d) δ 7.72 (d, J=8.2 Hz, 2H), 7.30 (d, J=8.0 Hz, 2H), 5.19 (d, J=8.2 Hz, 1H), 4.01-3.92 (m, 3H), 2.42 (s, 3H), 2.20 (t, J=7.4 Hz, 2H), 1.80 (pent, J=7.2 Hz, 2H), 1.45 (s, 9H), 1.39 (d, J=7.2 Hz, 3H).

2-(methylamino)ethyl tosyl-L-alaninate 9a: A solution of 8a (1.40 g, 3.49 mmol, 1 equiv) in dry CH₂Cl₂ (10 ml) was added HCl (2.6 ml, 10.48 mmol, 3 equiv, 4M solution in dioxane), and the resulting reaction was stirred at room temperature for 3 hours. The reaction was concentrated and triturated with methanol and diethyl ether, the resulting precipitates were collected through filtration to give the title compound 9a (R=methyl) as hydrochloride salt (1.08 g, 92%). ¹H NMR (400 MHz, DMSO-d6) δ 8.86 (br, 2H), 8.28 (br, 1H), 7.67 (d, J=8.2 Hz, 2H), 7.40 (d, J=8.0 Hz, 2H), 4.11 (m, 1H), 4.02 (m, 1H), 3.92 (m, 1H), 3.07 (m, 2H), 2.56 (s, 3H), 2.39 (s, 3H), 1.20 (d, J=7.2 Hz, 3H). LCMS (m/z) 301.2 (M+1); RT (Method 2, std LCMS method), 0.73 min.

Compound 9b (R=hydrogen) was prepared using the reaction condition described above to give the title compound 9b (R=hydrogen) (1.3 g, 92%) as hydrochloride salt. ¹H NMR (400 MHz, DMSO-d6) δ 8.28 (br, 4H), 7.68 (d, J=8.0 Hz, 2H), 7.39 (d, J=8.1 Hz, 2H), 4.05 (m, 1H), 3.94 (m, 2H), 2.94 (m, 2H), 2.38 (s, 3H), 1.21 (d, J=7.2 Hz, 3H). LCMS (m/z) 287.1 (M+1); RT (Method 2, std LCMS method), 0.74 min.

2,2-dimethyl-4-((tosyl-L-alanyl)oxy)butanoic acid 12a: A solution of 11a (1.11 g, 2.68 mmol, 1 equiv) in CH₂Cl₂ (9 ml) was added TFA/CH₂Cl₂ (v:v/1:1, 9 ml) at 0° C., and the resulting reaction was raised to room temperature and stirred for 3 hours. The reaction was concentrated and purified by Biotage flash chromatography (gradient elution, 0 to 75% EtOAc in hexanes) to obtain the title compound 12a (R=methyl) (930 mg, 97%) as colorless gel. ¹H NMR (400 MHz, Chloroform-d) δ 7.74 (d, J=8.2 Hz, 2H), 7.30 (d, J=8.1 Hz, 2H), 5.38 (d, J=8.8 Hz, 1H), 4.09-3.96 (m, 3H), 2.42 (s, 3H), 1.96-1.82 (m, 2H), 1.32 (d, J=7.2 Hz, 3H), 1.24 (s, 3H), 1.21 (d, 3H). LCMS (m/z) 358.1 (M+1); RT (Method 2, std LCMS method), 1.15 min.

Compound 12b (R=hydrogen) was prepared using the reaction condition described above to give the title compound 12b (R=hydrogen) (1.46 g, 87%) as colorless gel. ¹H NMR (400 MHz, Chloroform-d) δ 7.74 (d, J=8.2 Hz, 2H), 7.30 (d, J=8.4 Hz, 2H), 5.35 (br, 1H), 5.28 (d, J=8.6 Hz, 1H), 4.05-3.95 (m, 3H), 2.42 (s, 3H), 2.37 (t, J=7.2 Hz, 2H), 1.94-1.84 (m, 2H), 1.37 (d, J=7.2 Hz, 3H). LCMS (m/z) 330.1 (M+1); RT (Method 2, std LCMS method), 1.02 min.

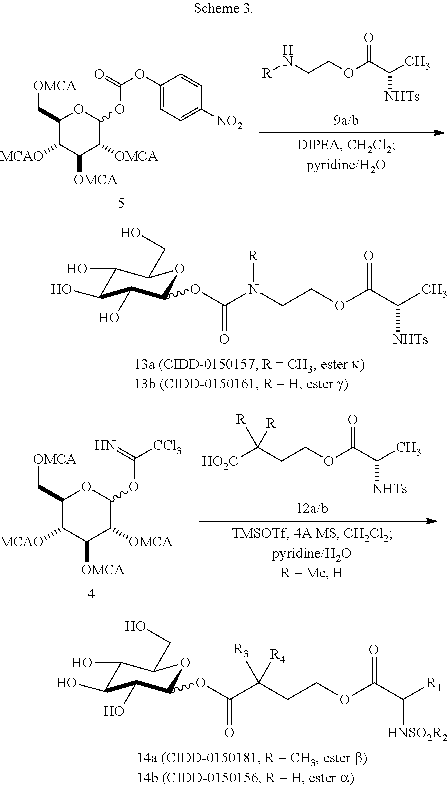

Referring to Scheme 3 above, a compound of the formula 5 is reacted with the glycosyl acceptor such as compounds 9a/b in the presence of an organic base such as DIPEA or triethylamine in a solvent such as dichloromethane at a temperature from 0° C. to room temperature for a time from 1 hour to 24 hours. Alternatively, a compound of the formula 4 is reacted with the glycosyl acceptor such as compounds 12a/b in the presence of an organic acid such as TMSOTf and molecular sieves in a solvent such as dichloromethane at a temperature from 0° C. to room temperature for a time from 1 hour to 24 hours. R depicted in scheme 3 is individually selected from groups such as hydrogen, methyl and other alkyl substituents. Finally, removal of the MCA protecting groups is accomplished in the presence of a tertiary amine or an organic amine base such as DIPEA or pyridine in a solvent such as water at a temperature from 0° C. to room temperature for a time from 1 hour to 24 hours.

2-(methyl((((3R,4S,5S,6R)-3,4,5-trihydroxy-6-(hydroxymethyl)-tetrahydro-2H-pyran-2-yl)oxy)carbonyl)amino)ethyl tosyl-L-alaninate 13a (CIDD-0150157): A solution of 5 (475 mg, 0.73 mmol, 1 equiv) and 9a (R=methyl) (245 mg, 0.73 mmol, 1 equiv) in dry CH₂Cl₂ (5 ml) was added DIPEA (188 mg, 1.46 mmol, 2 equiv), and the resulting reaction was stirred at room temperature for 24 hours. The reaction was concentrated and added pyridine/water (v:v/1:1, 10 ml), and the reaction mixture was stirred at room temperature for 48 hours. The reaction was concentrated and azerotropically removing the residual water by co-evaporating with toluene for couple times under reduced pressure, and the material was purified by Biotage flash chromatography (gradient elution, 0 to 25% MeOH in CH₂Cl₂) to obtain the title compound 13a (CIDD-0150157, R═CH₃, glucosyl ester κ) (225 mg, 50%) as colorless gel. ¹H NMR (400 MHz, Methanol-d4) δ 7.72 (d, J=8.1 Hz, 2H), 7.37 (d, J=8.0 Hz, 2H), 5.99 (d, J=3.3 Hz, OH), 4.14-3.91 (m, 3H), 3.78-3.66 (m, 3H), 3.64-3.41 (m, 5H), 2.43 (s, 3H), 1.37 (d, J=6.4 Hz, 3H), 1.28 (d, J=6.8 Hz, 3H). LCMS (m/z) 505.8 (M−1); RT (Method 2, std LCMS method), 0.80 min

Compound 13b (CIDD-0150161, R═H, glucosyl ester γ) was prepared using the reaction condition described above with the corresponding compound 9b (R=hydrogen) to give the title compound 13b (R=hydrogen) (222 mg, 62%) as colorless foams. ¹H NMR (400 MHz, Methanol-d4) δ 7.72 (d, J=7.9 Hz, 2H), 7.37 (d, J=7.9 Hz, 2H), 5.97 (d, J=3.4 Hz, 1H), 3.97-3.82 (m, 4H), 3.77-3.62 (m, 4H), 3.27-3.20 (m, 3H), 2.42 (s, 3H), 1.29 (d, J=7.2 Hz, 3H). LCMS (m/z) 491.7 (M−1); RT (Method 2, std LCMS method), 0.79 min

(2S,3R,4S,5S,6R)-3,4,5-trihydroxy-6-(hydroxymethyl)tetrahydro-2H-pyran-2-vi 2,2-dimethyl-4-((tosyl-L-alanyl)oxy)butanoate 14a (CIDD-0150181): To a flame-dried round-bottom flask with activated 4A molecular sieves was charged with compound 4 (705 mg, 1.12 mmol, 1 equiv) and 12a (R=methyl) (440 mg, 1.23 mmol, 1.1 equiv) and dissolved the mixture in dry CH₂Cl₂ (12 ml). The mixture was cooled to 0° C. and added TMSOTf (50 mg, 0.22 mmol, 0.2 equiv), and the reaction was stirred at this temperature gradually raised to room temperature without replenishing the cooling bath for the next 24 hours. The reaction was quenched by adding saturated NaHCO₃ (aq.) (20 ml) and extracted with CH₂Cl₂ (2×20 ml). The combined organic extracts were dried over Na₂SO₄, filtered and concentrated under reduced pressure. The crude material was diluted with pyridine/water (v:v/1:1, 10 ml), and the reaction mixture was stirred at room temperature for 48 hours. The reaction was concentrated and azerotropically removing the residual water by co-evaporating with toluene for couple times under reduced pressure, and the material was purified by Biotage flash chromatography (gradient elution, 0 to 18% MeOH in CH₂Cl₂) to obtain the title compound 14a (CIDD-0150181, R═CH₃, glucosyl ester β) (327 mg, 56%) as colorless foam. ¹H NMR (400 MHz, Methanol-d4) δ 7.72 (d, J=8.3 Hz, 2H), 7.36 (d, J=8.0 Hz, 2H), 5.45 (d, J=8.0 Hz, 1H), 4.00-3.78 (m, 4H), 3.76-3.64 (m, 1H), 3.45-3.34 (m, 4H), 2.43 (s, 3H), 1.86-1.71 (m, 2H), 1.27 (d, J=7.2 Hz, 3H), 1.21 (s, 3H), 1.20 (s, 3H). LCMS (m/z) 518.8 (M−1); RT (Method 2, std LCMS method), 0.92 min

Compound 14b (CIDD-0150156, R═H, glucosyl ester α) was prepared using the reaction condition described above with the corresponding compound 12b (R=hydrogen) to give the title compound 14b (R=hydrogen) (126 mg, 41%) as colorless foam. ¹H NMR (400 MHz, Methanol-d4) δ 7.71 (d, J=8.2 Hz, 2H), 7.37 (d, J=8.1 Hz, 2H), 5.49 (d, J=8.2 Hz, 1H), 3.98-3.82 (m, 4H), 3.68 (dd, J=12.0, 4.9 Hz, 1H), 3.45-3.35 (m, 4H), 2.42 (s, 3H), 2.43-2.37 (m, 2H), 1.81 (pent, J=6.9 Hz, 2H), 1.29 (d, J=7.2 Hz, 3H). LCMS (m/z) 490.8 (M−1); RT (Method 2, std LCMS method), 0.85 min

Electrochemical Testing. The four glucosyl esters α, β, γ, and κ (Scheme 3) were tested for the determination of LE in synovial (joint) fluid and urine samples:

$$ester α , β , γ , or κ {\to }^{LE} glucose \qquad ( 1 )$$

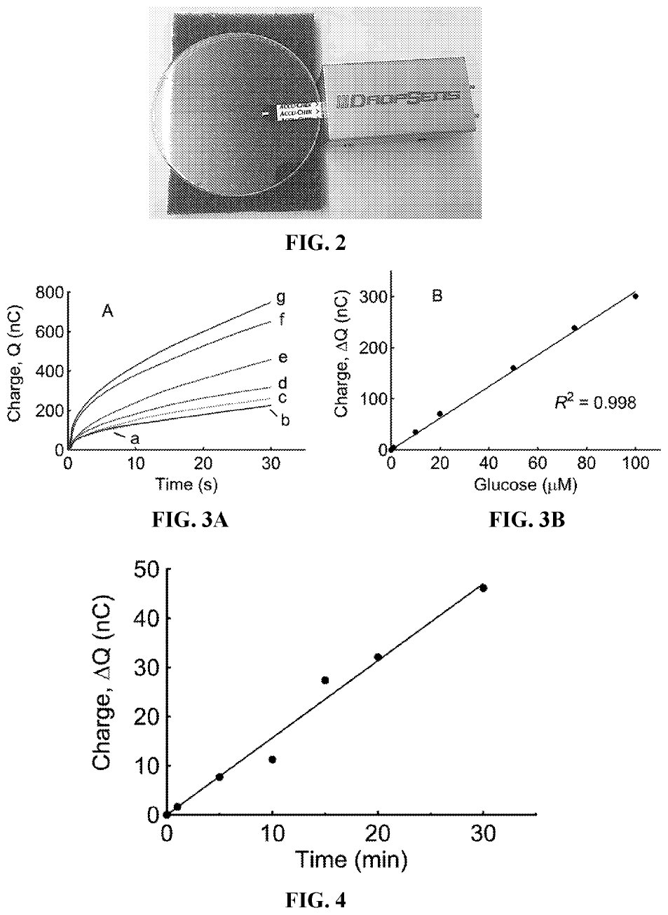

The samples were incubated with each ester to release glucose in the direct proportion to the enzymatic activity of LE in a sample. The released glucose was then detected at a commercial glucose test strip. A laboratory potentiostat was used to read the glucose strip. The strip was inserted in an electronic holder to facilitate the measurements (FIG. 2). Such a strip-potentiostat measuring system provided a much lower limit of glucose detection (5.0 μM, S/N=3; FIG. 3A) and a linear range at least up to 100 μM glucose (FIG. 3B), which allowed for a much shorter (minutes) incubation times (FIG. 4).

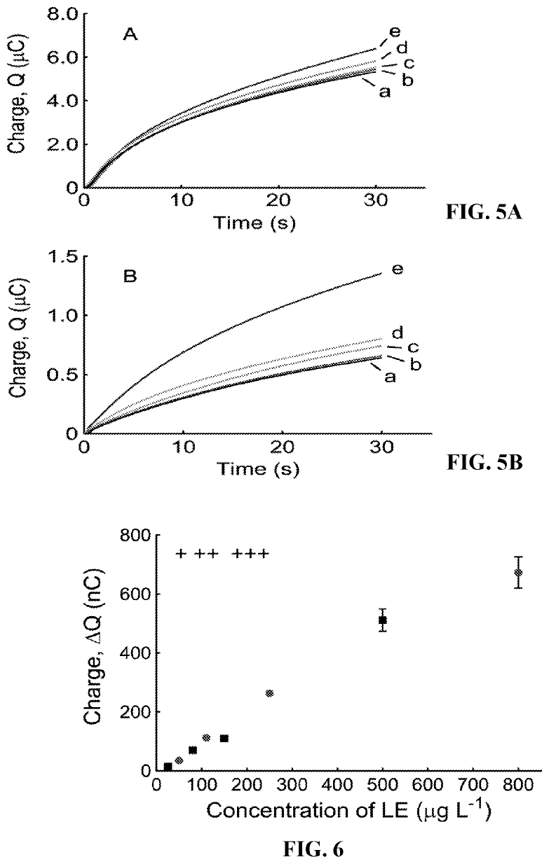

FIG. 5 shows the chronocoulometric plots that were recorded with the strip-potentiostat system after only a 5-min sample incubation with ester α. The charge flowing through a glucose strip in contact with a LE-free synovial fluid sample (panel A, trace a) was much larger than the corresponding charge for urine (panel B, trace a). Despite this difference, the rise in the LE content of either sample caused the increase in a charge flowing through the contacting glucose test strip.

FIG. 6 shows the direct correlation between the glucose strip's signal (ΔQ) and the concentration of LE in synovial fluid and urine. It illustrates several useful features including the:
a. distinction of different LE contents within a one-color zone of a colorimetric LE strip
b. short sample incubation time (e.g. 5 min instead of hours)
c. clinically relevant LE range from 25 up to at least 800 μg L¹
d. one convergent calibration plot for different real-life samples
e. linear “signal vs. concentration” plot due to the prevalence of zero-order kinetics
f. quantitative assessment of infection irrespective of sample transparency (opaque, colored, bloodied)

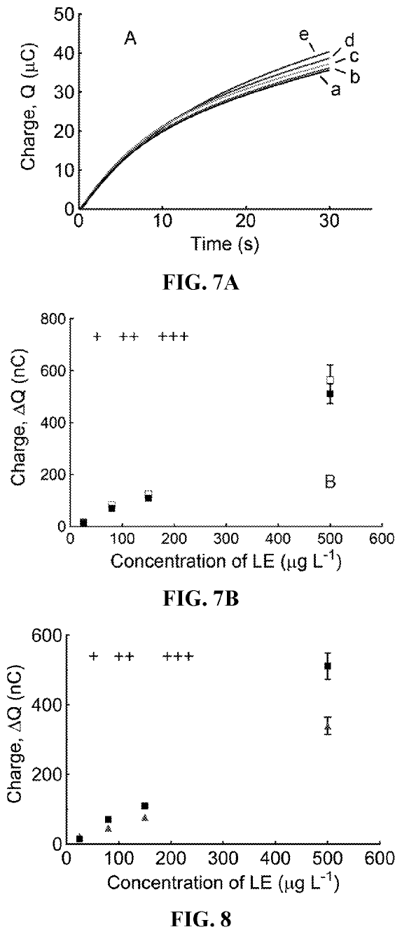

FIG. 7A shows the chronocoulometric plots that were recorded for urine samples containing very high concentration of native glucose (18 mM). As predicted, the charge Q flowing through a glucose strip in contact with such a sample was very high (trace a). Nevertheless, the ester-glucose strip system could detect the extra glucose produced by LE present in such samples (traces b-e). FIG. 7B shows that the proposed differential measurement based on ΔQ could reliably account for the presence of native glucose in a sample as indicated by the overlap of data points recorded in the presence and absence of glucose in a sample.

Analysis of LE with Ester γ. When compared to α, the ester γ required a longer incubation time (10 min) to yield enough glucose (reaction 1) to be above the detection limit of the potentiostat/glucose strip system (5 μM). FIG. 8 shows that γ also gave a ˜10% lower calibration slope for LE in urine. The longer sample incubation time and lower calibration slope could be ascribed to the slower kinetics of the enzymatic reaction of LE with ester γ. The slower kinetics could be attributed to the more proteolytically stable carbamate functionality of ester γ (Scheme 3).

The LE-triggered release of glucose from the glucosyl esters provides a relatively rapid method for the screening of human synovial (joint) fluid and urine samples for infections. Such esters, together with commercial glucose strips and a laboratory potentiostat, offer a superior signal resolution and the ability to quantify an infection enzyme LE in real-life samples regardless of their state (opaque, colored, bloodied) and the presence of potentially interfering species including native glucose. Such a screening of patient samples has a potential to facilitate clinical decisions about the use of antibiotics e.g. in the case of urinary tract and periprosthetic joint infections; the latter being one of the most devastating and costly complications of joint reconstruction/replacement (Wang et. al., Med. Sci. Monit. 2017, 23, 353-358; Kurtz et al., J. Arthroplasty 2012, 27, 61-65; Peel et al., J. Hosp. Infect. 2013, 85, 213-219.

1. A glucosyl ester having a chemical formula of Formula I or a salt or solvate thereof: 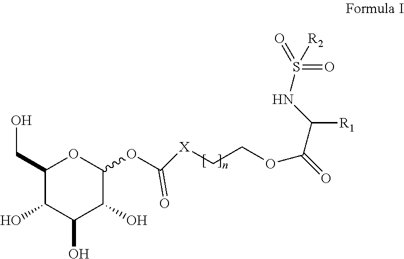
wherein, X is O, CR₃R₄ or NR₅, and wherein R₁, R₂, R₃, R₄ and R₅ are independently hydrogen, a C₁ to C₈ alkyl, a substituted C₁ to C₈ alkyl, a C₃ to C₇ cycloalkyl, a substituted C₃ to C₇ cycloalkyl, a heteroalkyl, a substituted heteroalkyl, a heterocycle, a substituted heterocycle, an aryl, a substituted aryl, a fused aryl, a substituted fused aryl, a heteroaryl, or a substituted heteroaryl.

2. The glucosyl ester of claim 1, wherein X is CR₃R₄, R₁, R₃, and R₄ are independently hydrogen, or a C₁ to C₈ alkyl, and R₂ is an aryl or a substituted aryl.

3. The glucosyl ester of claim 2, wherein R₁ is methyl, R₃ and R₄ are independently hydrogen or methyl, and R₂ is a substituted aryl.

4. The glucosyl ester of claim 1 having the chemical formula of formula II 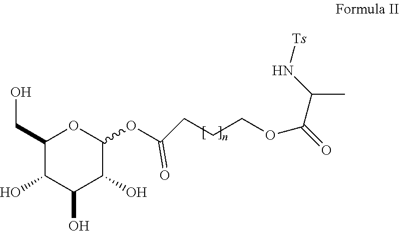
wherein Ts is tosyl group.

5. The glucosyl ester of claim 1 having the chemical formula of formula III 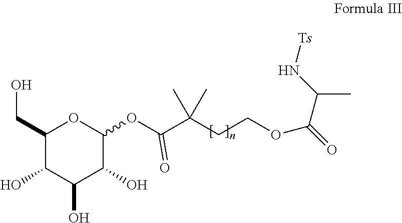
wherein Ts is tosyl group.

6. The glucosyl ester of claim 1, wherein X is NR₅, R₁ and R₅ are independently hydrogen, or a C₁ to C₈ alkyl, and R₂ is an aryl or a substituted aryl.

7. The glucosyl ester of claim 1 having the chemical formula of formula IV 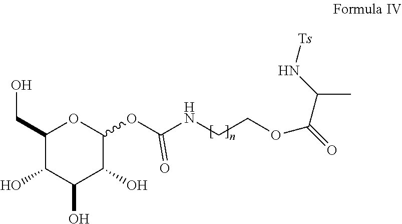
wherein Ts is tosyl group.

8. The glucosyl ester of claim 1 having the chemical formula of formula V 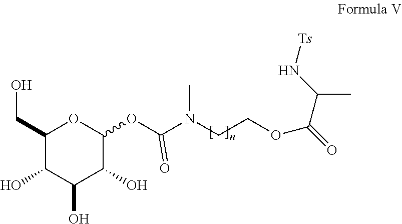
wherein Ts is tosyl group.

9. A method for detecting leukocyte esterase (LE) activity in a sample comprising:
contacting the sample with a glucosyl ester or a salt or solvate thereof of claim 1 forming a test sample; and
detecting cleavage of the glucosyl ester by LE in the test sample.

10. The method of claim 9, wherein cleavage of the glucosyl ester by LE is determined by measuring concentration of glucose liberated in the test sample by cleavage of the glucosyl ester by LE.

11. The method of claim 10, wherein the glucose in the test sample is measured by electrochemical detection.

12. The method of claim 9, wherein the initial glucosyl ester concentration in the test sample is above 5 mg/L.

13. A method for treating an infection in a subject, the method comprising:
contacting a sample obtained from the subject with a glucosyl ester or a salt or solvate thereof of claim 1 to form a test sample;
determining glucose concentration in the test sample; and
administering a treatment of the infection to the subject if the glucose concentration in the test sample is elevated with respect to a non-infected control.

14. The method of claim 13, wherein the subject is a human.

15. The method of claim 13, wherein the sample is a blood, plasma, serum, tears, urine, synovial (joint) fluid or saliva sample.

16. The method of claim 13, wherein the infection is a urinary tract infection, or periprosthetic joint infection.

17. A composition comprising the glucosyl ester of claim 1.

18. A kit comprising the glucosyl ester of claim 1.

19. The kit of claim 18, further comprising a glucose detection component.
